(19) United States
(12) Patent Application Publication
(10) Pub. No.: US 2005/0254999 A1
(43) Pub. Date: Nov. 17, 2005

(54) Clinical specimen processing apparatus and clinical specimen processing system

(75) Inventor: Hideyuki Higuchi, Kobe-shi (JP)

Correspondence Address: BRINKS HOFER GILSON & LIONE, P.O. BOX 10395, CHICAGO, IL, 60610, US

(73) Assignee: Sysmex Corporation

(21) Appl. No.: 11/127,644

(22) Filed: May 12, 2005

(30) Foreign Application Priority Data
May 12, 2004 (JP) 2004-142639

(51) Int. Cl. G01N 33/00

(52) U.S. Cl. 422/63; 422/68.1

(57) ABSTRACT

Clinical specimen processing apparatuses and clinical specimen processing systems are described, which are connectable to a management apparatus over a network.

This application claims priority under 35 U.S.C. § 119 to Japanese Patent Application No. 2004-142639 filed May 12, 2004, the entire content of which is hereby incorporated by reference.

### FIELD OF THE INVENTION

The present invention relates to a clinical specimen processing apparatus and clinical specimen processing system, and specifically relates to a clinical specimen processing apparatus and clinical specimen processing system, which are connectable to a maintenance management apparatus and user management apparatus over a network.

### BACKGROUND

There are well known conventional automatic analyzers which process measurements over time, assay results, or analyzer abnormalities, and measurement operation abnormalities by means of electronic mail, which is then transmitted to a specific destination (for example, refer to Japanese Laid-Open Patent Publication No. 10-308737).

Japanese Laid-Open Patent Publication No. 10-308737 discloses an automatic analyzer which, when performing normal assays, transmits device measurements over time and assay results by electronic mail to a predetermined destination, such as the personal computer of the user of the analyzer, by a predetermined timing, and when abnormalities are generated in the device, transmit data indicating the content of the abnormality of the device by electronic mail to a predetermined destination, such as the personal computer of the user or the like, rapidly without waiting the predetermined timing. In this way the, as described in Japanese Laid-Open Patent Publication No. 10-308737, the user of the automatic analyzer is quickly alerted to the abnormality when an abnormality occurs in the automatic analyzer.

Smear preparation devices which prepare blood specimen smears, and hemocyte analyzers (blood analyzers) which analyze blood specimens to determine the number of blood corpuscles, hematocrit, or hemoglobin and the like, are known types of clinical specimen processing apparatuses. Since these devices must provide accurate and rapid specimen processing information, such as assay results and smear acceptability, any abnormality must be quickly reported to the maintenance company when the device stops working due to an abnormal occurrence in the device. Furthermore, since specimen processing information may include private information, such information must be handled with great care.

When the art disclosed in Japanese Laid-Open Patent Publication No. 10-308737 is applied to clinical specimen processing apparatuses and an abnormality is generated in the clinical specimen processing apparatus, data representing the content of the abnormality is transmitted to a predetermined destination, such as the personal computer of the user of the automatic analyzer, with the result that the user may be inconvenienced by the necessity of contacting the company which maintains the analyzer and providing the abnormality information. Therefore, a disadvantage is that there is an increased load on the user when abnormalities occur.

When the art disclosed in Japanese Laid-Open Patent Publication No. 10-308737 is applied to clinical specimen processing apparatuses, the predetermined destination to which the abnormality information is sent to is considered to be the maintenance company rather than the user. In this case, however, since the specimen processing information, such as the assay result which includes personal information, is also transmitted to the maintenance company with a predetermined timing, a new disadvantage is created, that is, the patient's private information is dispersed to the maintenance company.

U.S. Pat. No. 6,629,060 discloses art for transmitting predetermined status information indicating the historical operating status of an analyzer over a network to a management apparatus.

### SUMMARY

One object of the present invention is to eliminate the previously described disadvantages by providing a clinical specimen processing apparatus and clinical specimen processing system which do not require the user to send information concerning abnormalities of the apparatus to a maintenance company, and prevent the private information included in the assay data of a specimen from being sent out.

A first aspect of the present invention is a clinical specimen processing apparatus connectable over a network to a first management apparatus and a second management apparatus, the clinical specimen processing apparatus including a specimen processing apparatus for performing predetermined processing of clinical specimens, and a data processing apparatus comprising a first transmission means for transmitting status information representing the historical operating status of the specimen processing apparatus to the first management apparatus with a predetermined timing, a second transmission means for transmitting emergency information within the status information to the first management apparatus without waiting for the predetermined timing, and a third transmission means for transmitting specimen processing information obtained by predetermined processing of a clinical specimen to the second management apparatus.

A second aspect of the present invention is a clinical specimen processing apparatus connectable over a network to a management apparatus, the clinical specimen processing apparatus including a specimen processing apparatus for performing predetermined processing of clinical specimens, and a data processing apparatus comprising a first transmission means for transmitting a first electronic mail of first information relating to the specimen processing apparatus in a predetermined format to the management apparatus with a predetermined timing; and a second transmission means for transmitting a second electronic mail of predetermined second information within the first information in a format which is different from the first electronic mail to the management apparatus without waiting for the predetermined timing.

A third aspect of the present invention is a clinical specimen processing system connectable over a network to a management apparatus, the clinical specimen processing system including a first specimen processing apparatus for performing predetermined processing of clinical specimens, a second specimen processing apparatus for performing predetermined processing of clinical specimen, and a data processing apparatus comprising a first transmission means for transmitting status information representing the historical operating status of the first specimen processing apparatus to the management apparatus with a predetermined timing, a second transmission means for transmitting emergency information within the status information of the first specimen processing apparatus to the management apparatus without waiting for the predetermined timing, and a third transmission means for transmitting status information representing the historical operating status of the second specimen processing apparatus to the management apparatus with a predetermined timing.

### BRIEF DESCRIPTION OF THE DRAWINGS

FIG. 1 is a top view of the general structure of the clinical specimen processing apparatus of the first embodiment;

FIG. 2 is a perspective view of a sample rack used in the clinical specimen processing apparatus of the first embodiment shown in FIG. 1;

FIG. 3 is a flow chart describing the information transmission operation of the blood analyzer of the first embodiment shown in FIG. 1;

FIG. 4 is a flow chart illustrating the information transmission operating on the specimen smear preparation apparatus side in the first embodiment shown in FIG. 1;

FIG. 5 is a flow chart illustrating the operation of each apparatus including error generation by the specimen smear preparation apparatus, specimen smear preparation apparatus when starting shutdown, personal computer, and maintenance management apparatus in the first embodiment shown in FIG. 1;

FIG. 6 is a flow chart illustrating the information transmission operation on the blood analyzer side in a second embodiment;

FIG. 7 is a top view showing the general structure of the clinical specimen processing apparatus of a third embodiment;

FIG. 8 is a structural diagram showing the content of electronic mail including emergency information of the specimen smear preparation apparatus in the first embodiment;

FIG. 9 is a structural diagram showing the content of electronic mail including operating information of the specimen smear preparation apparatus in the first embodiment;

FIG. 10 is a table showing the content of the error log in the attached file of FIG. 9;

FIG. 11 is a table showing the content of the operation count in the attached file of FIG. 9; and

FIG. 12 is a table showing the content of the maintenance parts and replacement parts log in the attached file of FIG. 9.

### DETAILED DESCRIPTION OF THE PREFERRED EMBODIMENTS

The preferred embodiments of the present invention are described hereinafter with reference to the drawings.

### First Embodiment

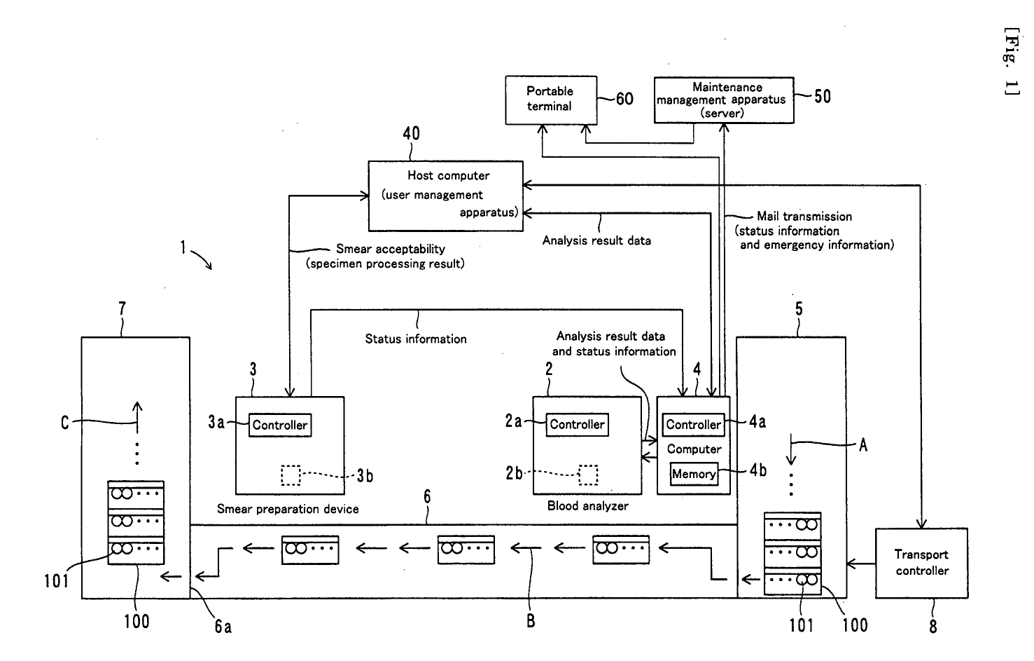

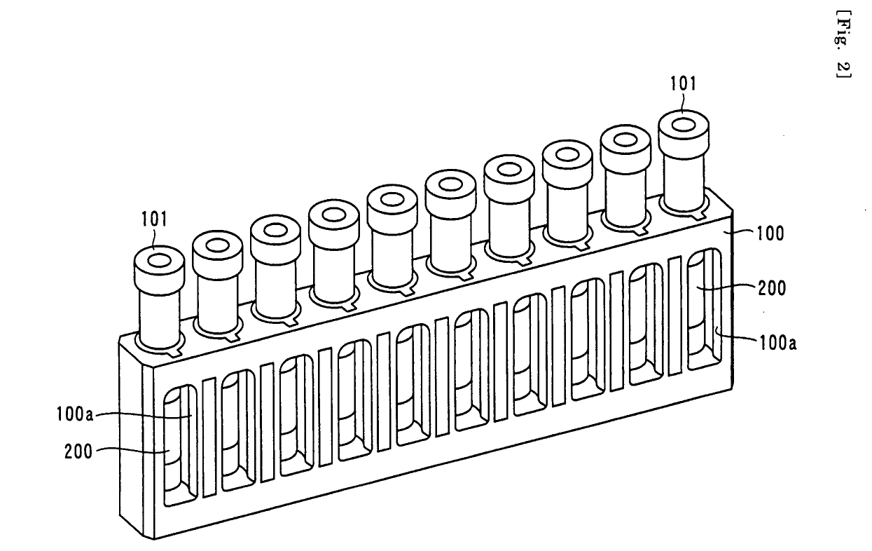

FIG. 1 is a top view showing the general structure of a clinical specimen processing apparatus including a blood analyzer and a specimen smear preparation apparatus in a first embodiment of the present invention. FIG. 2 is a perspective view of a sample rack used in the clinical specimen processing apparatus shown in FIG. 1.

The general structure of the clinical specimen processing apparatus 1 of the first embodiment is described below with reference to FIGS. 1 and 2. As shown in FIG. 1, the clinical specimen processing apparatus 1 of the first embodiment is provided with a blood analyzer 2, specimen smear preparation device 3, personal computer 4, loader 5 with a sample rack 100 installed therein, conveyer 6 for transporting the sample rack 100, unloader 7 for accommodating the used sample racks 100, and transport controller 8 for controllably driving the loader 5, conveyer 6, and unloader 7. Furthermore, the blood analyzer 2 and specimen smear preparation device 3 are arranged along the conveyer 6.

In the first embodiment, the personal computer 4 is connected over a network to a host computer (user management apparatus) 40 installed on the user side, maintenance management apparatus (server) 50 installed externally at the maintenance company, and portable terminal 60. Furthermore, the maintenance management apparatus (server) 50 is connected over the network to the portable terminal 60. The clinical specimen processing system is configured by the host computer (user management apparatus) 40, maintenance management apparatus (server) 50, and clinical specimen processing apparatus 1. The network may be a special network such as a telephone line, internet, intranet, or a network such as a local area network (LAN) and the like. Furthermore, the specimen smear preparation device 3 and transport controller 8 are also connected to the host computer 40 over the network. The host computer 40 is provided to perform information exchange and issue instructions to the specimen smear preparation device 3, personal computer 4, and transport controller 8. The personal computer 4, specimen smear preparation device 3, and blood analyzer 2 are connected by respective communication cables.

A plurality of sample racks 100 are used in the clinical specimen processing apparatus 1 of the first embodiment. A plurality of sample containers 101 respectively containing blood samples are accommodated in each sample rack 100, as shown in FIG. 2. A barcode label 200 provided with the sample identification information (ID) is adhered to each sample container 101. A window 100a is provided on the side surface of the sample rack 100 so as to allow the barcode label 200 adhered on the sample container 101 to be read.

The blood analyzer 2 is provided to collect and analyze the sample within the sample container 101. In the first embodiment, the blood analyzer 2 includes a controller 2a which has the function of transmitting the analysis results and status information of the blood analyzer 2 to the personal computer 4, and a barcode reader 2b for reading the barcode of the sample containers 101. The controller 2a configured by a CPU, RAM, ROM and the like.

The specimen smear preparation device 3 is installed in order to prepare specimen smears of samples which require specimen smear preparation based on the analysis result of the blood analyzer 2. The specimen smear preparation device 3 is connected to the personal computer 4 and the host computer 40. The specimen smear preparation device 3 includes a controller 3a which has the functions of transmitting specimen processing results such as whether or not the smear is acceptable to the host computer 40 and transmitting status information of the specimen smear preparation device 3 to the personal computer 4, and includes a barcode reader 3b for reading the bar codes on the sample containers 101. The controller 3a of the specimen smear preparation device 3 is configured by a CPU, RAM, ROM and the like.

The personal computer 4 includes a controller 4a which has the functions of transmitting status information of the blood analyzer 2 and status information of the specimen smear preparation device 3 to the maintenance management apparatus (server) 50 and transmitting analysis results of the blood analyzer 2 to the host computer 40, and a memory 4b of storing the analysis results and status information of the blood analyzer 2 and status information and the like of the specimen smear preparation device 3. The controller 4a of the personal computer 4 is configured by a CPU, RAM, and ROM and the like. The controller 4a of the personal computer 4 also stores programs sending and receiving and processing electronic mail.

The operation of the clinical specimen processing apparatus 1 of the first embodiment is described below with reference to FIGS. 1 through 5. First, the clinical chart information of the specimen provider (patient) is transmitted, for example, from an in-hospital database (not shown in the drawings) to the host computer 40 shown in FIG. 1. Then, when the clinical specimen processing apparatus 1 operates, the clinical specimen processing apparatus 1 starts when a sample rack 100 containing sample containers 101 is placed in the loader 5. After the sample rack 100 is transported in the arrow A direction to the input end of the conveyer 6 adjacent to the loader 5, the sample rack 100 is moved from the loader 5 to the conveyer 6. The first sample rack 100 is transported in the arrow B direction by the conveyer 6, and stopped in front of the blood analyzer 2. Then, the barcode of the first sample container is read by the barcode reader of the blood analyzer 2. Next, the blood analyzer 2 collects and analyzes the specimen in the sample container 101, and sends the barcode and analysis result data (analysis numerical value of each item, and abnormality message determined from these numerical values) to the personal computer 4. The personal computer 4 sends the barcode and analysis result to the host computer 40. The analyzed sample container 101 is moved in the arrow B direction a distance equal to the pitch between sample containers, and the specimen in the next sample container is analyzed. In this way the specimens are sequentially analyzed.

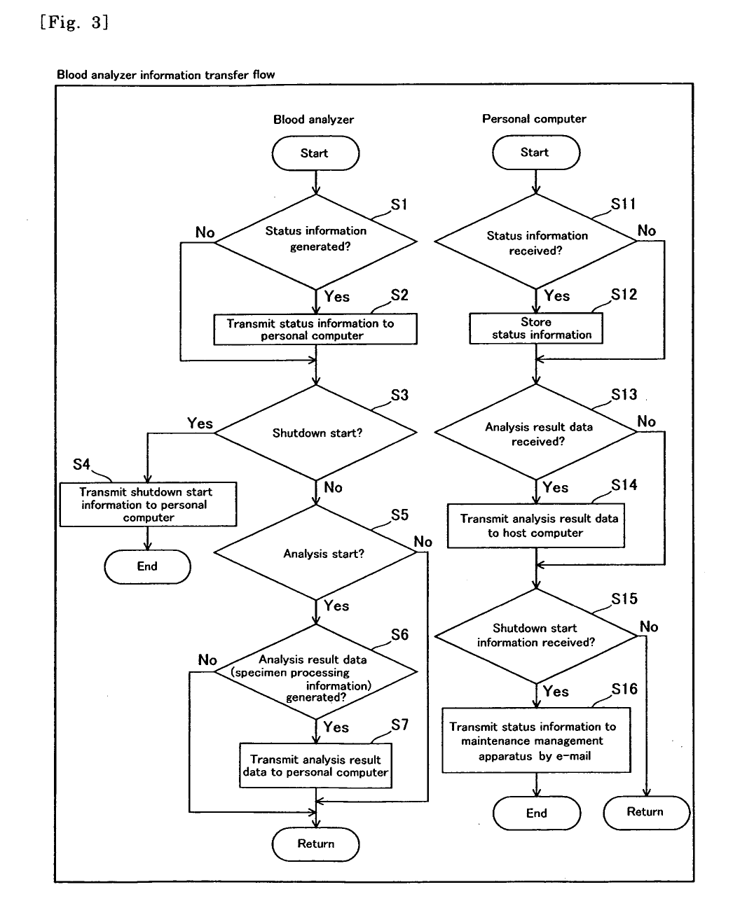

The information transmission operations of the host computer 40 on the blood analyzer 2 side and the maintenance management apparatus 50 are described in detail below with reference to FIG. 3. In step S1, the controller 2a of the blood analyzer 2 collects information from the sensors and the like which monitor the operation of the apparatus, and determines whether or not status information, such as error information indicating an operating abnormality, has been generated. When status information, such as error information indicating an operating abnormality, has been generated in step S1, the status information is transmitted from the blood analyzer 2 to the host computer 4 by the controller 2a of the blood analyzer 2 in step S2. Thereafter, a determination is made in step S3 as to whether or not the blood analyzer 2 has started a shutdown procedure. The routine advances to step S3 and makes this determination even when it is determined in step S1 that status information has not been generated. When shutdown has started in step S3, then the shutdown start information is transmitted from the blood analyzer 2 to the host computer 4 by the controller 2a of the blood analyzer 2 in step S4, and the process ends. When it is determined that shutdown has not started in step S3, then in step S5 a determination is made as to whether or not analysis has started. When it is determined in step S5 that analysis has started, then in step S6 a determination is made as to whether or not analysis result data (specimen processing information) have been generated. When it is determined that analysis result data have been generated, then in step S7 the analysis result data are transmitted from the blood analyzer 2 to the host computer 4 by the controller 2a of the blood analyzer 2. When it is determined in step S5 that analysis has not started, the routine returns to step S1, and the routine returns to step S1 even when it is determined in step S6 that analysis result data have not been generated.

The information transmission operation of the personal computer 4, among the information transmission operations on the blood analyzer 2 side, is described below with reference to FIG. 3. The controller 4a of the personal computer 4 determines whether or not status information of the blood analyzer 2, such as error information, has been received in step S11. When it is determined that status information of the blood analyzer 2, such as error information, has been received in step S11, the status information is stored in the memory 4b of the personal computer 4 in step S12. Thereafter, in step S13, a determination is made as to whether or not analysis result data of the blood analyzer 2 have been received. The routine advances to step S13 and this determination is made even when it is determined in step S11 that status information has not been received. When it is determined that analysis result data of the blood analyzer 2 have been received in step S13, the analysis result data (specimen processing information) is transmitted from the personal computer 4 over the network by the controller 4a of the personal computer 4 to the host computer 40. Thereafter, in step S15, a determination is made as to whether or not shutdown start information has been received. The routine advances to step S15 and this determination is made even when it is determined ion step S13 that analysis result information has not been received. When it is determined in step S15 that shutdown start information has been received, then in step S16 the status information of the blood analyzer 2 stored in the memory 4b of the personal computer 4 is transmitted by the controller 4a of the personal computer 4 to the maintenance management apparatus (server) 50 externally installed at the maintenance company, and the routine ends. When it is determined in step 15 that shutdown status information has not been received, the routine returns to step S11. In the first embodiment, the transmission of analysis result data from the personal computer 4 to the host computer 40 is accomplished by normal data communication without using electronic mail. The blood analyzer 2 and personal computer 4 repeat the process of steps S5˜S7 and S13˜S14 until the analysis of all specimens in the sample containers 101 of the sample rack 100 is completed.

In the first embodiment, electronic mail is used when status information of the blood analyzer 2 is transmitted from the personal computer 4 to the maintenance management apparatus (server) 50 in step S16. Specifically, the controller 4a of the personal computer 4 divides the status information of the blood analyzer 2 into a plurality of types, creates a plurality of attachment files respectively corresponding to the plurality of types, and attaches the plurality of attachment files to a single electronic mail, which is transmitted to the maintenance management apparatus (server) 50. The status information of the blood analyzer 2 may be, for example, an error log representing the status of abnormality if the blood analyzer 2, operation count representing the number of operations performed by the blood analyzer 2, and maintenance parts and replacement parts log representing the maintenance parts and replaced parts. Various information, such as device ID, date, time, error code and the like, may be included for each abnormality information in the attachment file when the error log represents the status of abnormality information of the blood analyzer 2. Furthermore, information such as the date, time, operation segment, and number of operations, may be included in the attachment file of status information of the operation count. The information transmission operation of the host computer 40 on the blood analyzer 2 side and the maintenance management apparatus 50 are performed.

The host computer 40 determines whether or not a specimen requires a smear preparation based on the analysis result data of the blood analyzer 2 transmitted from the personal computer 4 by the process of step S14. A specimen smear preparation may be necessary, for example, when the analysis result of the specimen analyzed by the blood analyzer 2 includes an abnormality message. Furthermore, a specimen smear preparation may be unnecessary, for example, when the analysis result of the specimen analyzed by the blood analyzer 2 does not include an abnormality message. A sample rack 100, which accommodates only specimens that the host computer 40 has determined do not require smear preparation, is moved past the specimen smear preparation device 3 to the unloader 7. The transport control of the sample rack 100 in this case is accomplished by the transport controller 8 controllably driving the conveyer 6 based on instructions from the host computer 40. In this way the sample rack 100 reaches the output end 6a of the conveyer 6. Then the unloader 7 adjacent to the output end 6a of the conveyer 6 moves the sample rack 7 in the arrow C direction to receive the rack.

A sample rack 100, which accommodates specimens that the host computer 40 has determined require smear preparation, is moved to the specimen smear preparation device 3. The transport control of the sample rack 100 in this case also is accomplished by the transport controller 8 controllably driving the conveyer 6 based on instructions from the host computer 40. Then the barcode of each sample container 101 is read by the barcode reader 3b of the specimen smear preparation device 3. The controller 3a of the specimen smear preparation device 3 transmits this barcode to the host computer 40, and queries the host computer 40 as to whether or not smear preparation is necessary for the specimen having that barcode. When the host computer 40 determines that the specimen with that barcode requires smear preparation, the specimen smear preparation device 3 collects the specimen from the sample container 101 which requires smear preparation, and prepares a blood specimen smear.

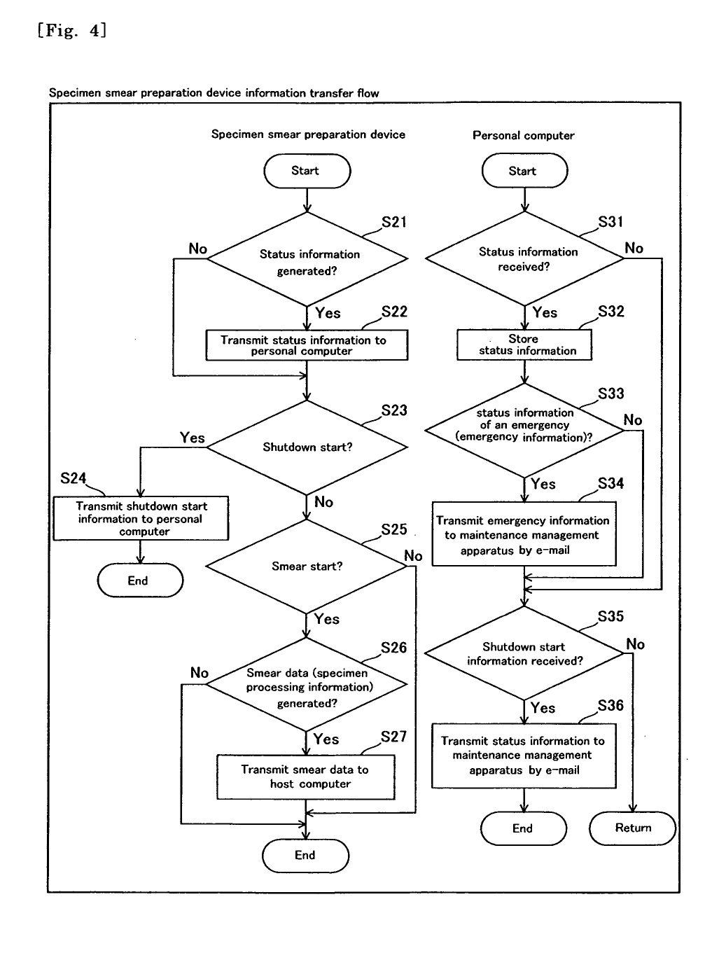

The information transmission operation of the host computer 40 on the specimen smear preparation device 3 side and the maintenance management apparatus 50 is described in detail below with reference to FIG. 4. The information transmission operation of the specimen smear preparation device 3 is described first below. In step S21 in FIG. 4, a determination is made as to whether or not status information has been generated, such as error information or the like, for the specimen smear preparation device 3. When it is determined that status information, such as error information or the like, has been generated for the specimen smear preparation device 3 in step S21, then in step S22 the status information is transmitted from the specimen smear preparation device 3 to the personal computer 4 by the controller 3a of the specimen smear preparation device 3. Thereafter, in step S23, a determination is made as to whether or not shutdown has started. The routine advances to step S23 and this determination is made even when it is determined in step S21 that status information has not been generated. When shutdown has started in step S23, then the shutdown start information is transmitted from the specimen smear preparation device 3 to the personal computer 4 by the controller 3a of the specimen smear preparation device 3, and the routine ends. When shutdown has not started in step S23, then in step S25 a determination is made as to whether or not the smear has started. When it is determined in step S25 that the smear has started, then in step S26 a determination is made as to whether or not acceptable smear data (specimen processing information) have been generated. When it has been determined that acceptable smear data (smear processing information) have been generated, then in step S27 the controller 3a of the specimen smear preparation device 3 transmits the smear data from the specimen smear preparation device 3 to the host computer 40. When it is determined in step S25 that smearing has not started, the routine returns to step S21, and when it is determined in step S26 that smear data (smear processing information) have not been generated, the routine also returns to step S21. In the first embodiment, the transmission of smear data from the specimen smear preparation device 3 to the host computer 40 is accomplished by normal data communication without using electronic mail.

The information transmission operation of the personal computer among the information transmission operations on the specimen smear preparation device 3 side is described below with reference to FIG. 4. In step S31, a determination is made as to whether or not status information, such as error information or the like, for the specimen smear preparation device 3 has been received. When it is determined in step S31 that status information, such as error information or the like, for the specimen smear preparation device 3 has been received, then the status information is stored in the memory 4b of the personal computer 4 in step S32. Then in step S33 a determination is made as to whether or not status information of the specimen smear preparation device 3 stored in the memory 4b of the personal computer 4 is status information of an emergency (emergency information). Status information of an emergency (emergency information) means error information indicating a situation which is difficult for a user to deal with, and from which recovery is impossible without a service visit.

The type of emergency information (error code) is set beforehand in the personal computer 4. When the existence of emergency information is determined in step S33, controller 4a of the personal computer 4 transmits the emergency information immediately, without waiting for shutdown, from the personal computer 4 over the network to the maintenance management apparatus (server) 50, in step S34. In the first embodiment, the emergency information of the specimen smear preparation device 3 is sent by electronic mail, and the information is sent in the text body of the electronic mail without creating an attached file. The body of the electronic mail in this case may include information such as, for example, the device ID, date, time, error code and the like.

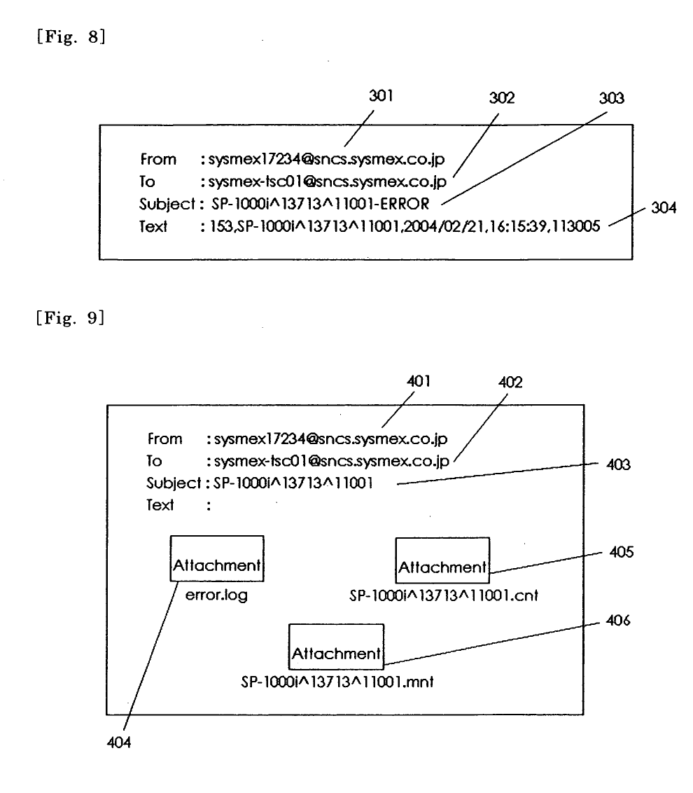

The specific form of the electronic mail including emergency information (emergency error mail) is shown in FIG. 8. The emergency error mail shown in FIG. 8 is configured by a transmission origin mail address 301, transmission destination mail address 302, subject header 303, and text body 304. The subject header 303 adds the text “ERROR” to the device ID so as to alert the reader that emergency information is included. The text body 305 includes a transmission number (153), device ID (SP-1000i″13713″11001), date of error generation (2004/02/21), time of error generation (16:15:39), and error code (113005). The error code 113005 represents a vacuum abnormality supplied from the vacuum source for creating a vacuum in the specimen smear preparation device 3. Since the device ID, date, time, and error code information are written directly in the text body, the maintenance company which receives the emergency error mail at the maintenance management apparatus (server) 50 can respond quickly to the apparatus abnormality. Furthermore, if service personnel of the ma9ntenance company is alerted of the emergency error mail via a portable terminal (notebook computer, portable telephone), the emergency error mail is extremely effective compared to electronic mail alters via attached files described later and which cannot be opened on some portable terminals since the device ID, data, time, and error code are written directly in the text body.

When it is determined in step S31 that status information such as error information of the specimen smear preparation device 3 has not been received, when it is determined in step S33 that status information of an emergency (emergency information) does not exist, when it is determined in step S35 that shutdown start information of the specimen smear preparation device 3 has not been received, then the routine returns to step S31. Furthermore, when it is determined in step S35 that shutdown start information has been received, the personal computer 4 which received the shutdown start information transmits the status information of the specimen smear preparation device 3 stored in the memory 4b of the personal computer 4 over the network to the maintenance management apparatus (server) 50 using electronic mail by means of the controller 4a of the personal computer 4.

In the transmission process of the status information during shutdown, all status information of the specimen smear preparation device 3, including the status information for emergency (emergency information) is transmitted from the personal computer 4 to the maintenance management apparatus (server) 50 over the network. Furthermore, the status information of the specimen smear preparation device 3 is transmitted by creating attachment files for each type of status information and sent as an attached file by electronic mail. The types of status information of the specimen smear preparation device 3 may be, for example, an error log representing the status history of abnormality information of the specimen smear preparation device 3, operation count representing the number of operations performed such as number of smears and number of stainings and the like, and maintenance parts and replacement parts log representing the maintenance parts and replaced parts. Various information, such as device ID, date, time, error code and the like, may be included for each abnormality information in the attachment file when the error log represents the status of abnormality information of the specimen smear preparation device 3. Furthermore, information such as the date, time, operation segment such as smear and staining, and number of operations, may be included in the attachment file of status information of the operation count.

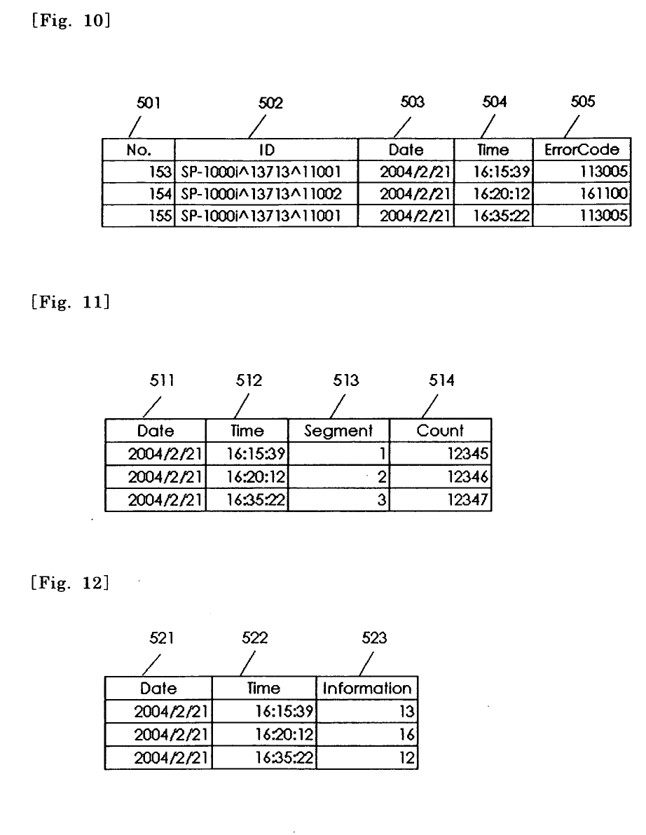

The specific form of the electronic mail with file attachment is shown in FIG. 9. The electronic mail shown in FIG. 9 is configured by a transmission origin mail address 401, transmission destination mail address 402, subject header 403, error log file attachment 404, operation count file attachment 405, and maintenance parts and replacement parts log file attachment 406. The subject header 403 has the device ID so as to alert the reader as to the object device. The error log file attachment 404 shown in FIG. 10 is a table configured by the transmission number (501), device ID number 502, error generation date 503, error generation time 504, and error code 505. The error code 113005 represents a pressure abnormality of the vacuum supplied from the vacuum source used by the device, and 161100 represents a blood suction abnormality. As shown in FIG. 11, the operation count file attachment 405 is a table configured by the operation generation date 511, operation generation time 512, segment 513, and operation number 514. In segment 513, 1 indicates the stain count, 2 represents the slide glass operation count, and 3 represents the total slide glass count. As shown in FIG. 12, the maintenance parts and replacement parts log file attachment 406 is a table configured by the replacement date 521, replacement time 522, and maintenance parts and replacement parts information 523. In the maintenance parts and replacement parts information 523, 13 indicates ink ribbon replacement, 16 indicates buffer solution replacement, and 12 indicates slide glass replacement. In this way the error frequency can be quickly ascertained and error classification can be easily understood by the error code 505 by allocating the content of the error to the error code. Furthermore, breakdowns can be estimated from the operation count by knowing the operation count of each part of the device by means of the segment 513. Which parts have been replaced can be known and next replacement dates can be estimated by the maintenance parts and replacement parts information 523. The file attachment electronic mail alert format allows a large quantity of data to be transmitted, and the file attachment can be read by commercial spreadsheet software, thereby being effective for processing large quantities of data.

In this way the information transmission operations are accomplished by the host computer 40 on the specimen smear preparation device 3 side and the maintenance management apparatus (server) 50.

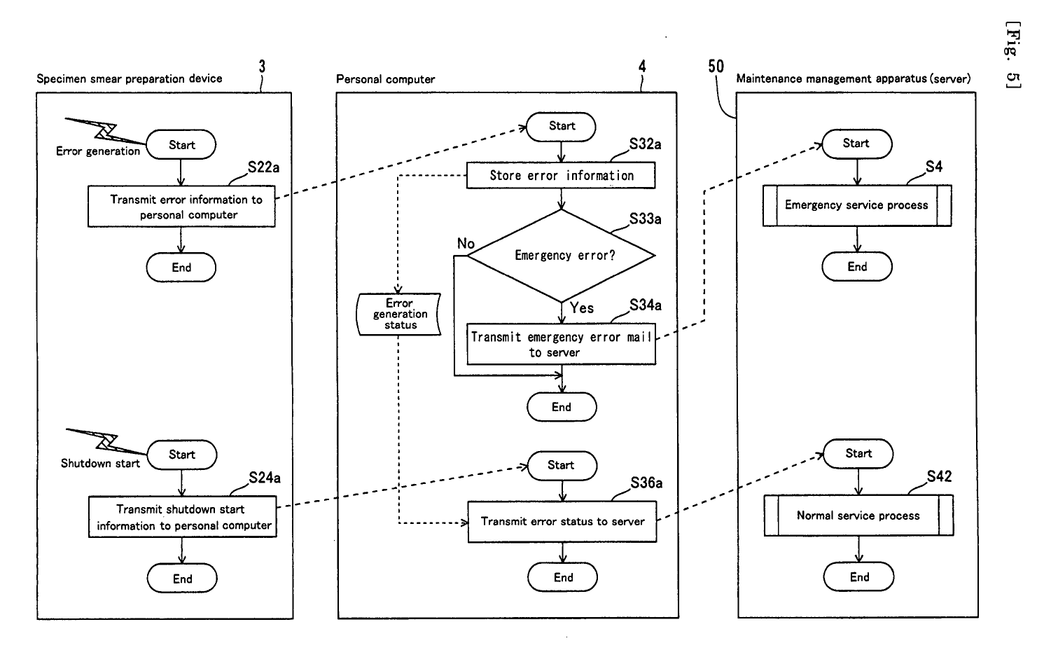

The operation of each device of the specimen smear preparation unit 3, personal computer 4, and maintenance management apparatus (server) 50 is described below when an error (abnormality) and shutdown operation are generated in the specimen smear preparation device 3 with reference to FIG. 5. The processes of steps S22a, S24a, S32a˜S34a, and S36a shown in FIG. 5 respectively correspond to steps S22, S24, S32˜S34, and S36 shown in FIG. 4. First, when an error (abnormality) is generated in the specimen smear preparation device 3, the error information is transmitted from the specimen smear preparation device 3 to the personal computer 4 by the controller 3a of the specimen smear preparation device 3. The error information is saved in the memory 4b of the personal computer 4, as shown in step S32a. Then, in step S33a, a determination is made as to whether or not the stored error information is an emergency error. An emergency error is defined as an error which is difficult for the user to deal with and which is difficult to recover from without a visit from service personnel of the maintenance company. The types of emergency error (error code) are set beforehand in the personal computer 4. A minor error which is recoverable from the user side is not an emergency error. Then a determination is made in step S33a by the personal computer 4 as to whether or not the stored error information is the error code of an emergency error set beforehand.

When the personal computer 4 determines that the error information is emergency error information in step S33a, then in step S34a an emergency error mail (electronic mail) is transmitted from the personal computer 4 to the maintenance management apparatus (server) 50 installed at the maintenance company. In this case the content of the emergency error is written in the text body of the electronic email and transmitted without attaching a file. When it is determined in step S33a that the information is not emergency information, the process ends directly.

The maintenance management apparatus (server) 50 which receives the emergency electronic mail performs an emergency service process in step S41. Specifically, service personnel from the maintenance company contacts the user by telephone or the like, and immediately visits the user to perform a recovery process on the specimen smear preparation device 3.

When shutdown has started in the specimen smear preparation device 3, the controller 3a of the specimen smear preparation device 3 transmits a shutdown start information from the specimen smear preparation device 3 to the personal computer 4 in step S24a. Then, the personal computer 4 which received to the shutdown start information transmits the error status stored in the memory 4b of the personal computer 4 to the maintenance management apparatus (server) 50 in step S36a. On the maintenance management apparatus (server) 50 side, the normal service process is performed in step S42. In the normal service process, the status information is checked for the day after the shutdown was performed to determine whether or not maintenance is required, and processes such as sending service personnel and the like are performed as necessary.

In the present embodiment, status information such as error information and the like of the specimen smear preparation device 3 is transmitted over a network from the personal computer 4 to the maintenance management apparatus (server) 50 installed externally at the maintenance company, and smear data (specimen processing information) of the specimen smear preparation device 3 is transmitted over the network from the specimen smear preparation device 3 to the host computer 40 on the user side, and therefore the user is not required to contact the maintenance company to send status information such as error information and the like of the specimen smear preparation device 3, and smear data which include private information such as patient ID and the like is prevented from being sent to the external maintenance company.

In the first embodiment, among the status information of the specimen smear preparation device 3, the emergency information (emergency error information) of an emergency which is difficult to recover from on the user side is transmitted over a network to the maintenance management apparatus (server) 50 immediately without waiting a predetermined timing, therefore when such an error, which is difficult to recover from, is generated on the user side, the maintenance company where the maintenance management apparatus (server) 50 is installed is immediately informed of the abnormality and quickly respond to perform maintenance. Furthermore, status information is transmitted periodically to the maintenance management apparatus (server) 50 by transmitting status information to the maintenance management apparatus (server) 50 during shutdown of the specimen smear preparation device 3.

In the first embodiment, status information and emergency information are transmitted from the personal computer 4 to the maintenance management apparatus 50 using electronic mail, and specimen processing information is transmitted from the personal computer 4 to the host computer 40 by normal data communication without using electronic mail, such that the transmission of information to the external maintenance management apparatus 50 can be accomplished by electronic mail, and transmission of information to the in-house host computer 40 can be accomplished by normal data communications without using electronic mail.

In the first embodiment, the controller 4a of the personal computer 4 transmits status information as a file attachment to electronic mail to the maintenance management apparatus (server) 50, and transmits emergency information in the text body of the electronic mail to the maintenance management apparatus (server) 50, such that the content of the emergency information can be readily understood simply by reading the text body of the electronic mail, thereby allowing the manager of the maintenance management apparatus (server) 50 to quickly respond. Furthermore, the content of status information which is not emergency information can be readily understood by opening the file attachment.

In the first embodiment, the controller 4a of the personal computer 4 divides the status information into a plurality of types, creates a plurality of file attachments corresponding to the respective plurality of types, and attaches the plurality of file attachments to a single electronic mail which is transmitted to the maintenance management apparatus (server) 50, thereby simplifying the status information transmission operation on the personal computer 4 side because a plurality of types of status information are transmitted in a single electronic mail. Management is also easy on the maintenance management apparatus (server) 50 side since the status information is classified into a plurality of types before management processes are performed on the maintenance management apparatus (server) 50 side by dividing the status information into a plurality of types, and creating and transmitting a plurality of file attachments corresponding to the respective plurality of types as file attachments to a single electronic mail.

In the first embodiment, the controller 4a of the personal computer 4 transmits status information not only of the specimen smear preparation apparatus 3 but also the status information of the blood analyzer 2 over a network to the maintenance management apparatus (server) 50, such that the status information of both the specimen smear preparation apparatus 3 and the blood analyzer 2 can be transmitted to the maintenance management apparatus (server) 50 using a single personal computer 4.

Although the electronic mail is transmitted to the maintenance management apparatus (server) 50 in the first embodiment, electronic mail also may be transmitted directly from the personal computer 4 to the portable terminal 60; furthermore, after once transmitting electronic mail to the maintenance management apparatus (server) 50 at the maintenance company, the electronic mail may be transmitting from the maintenance management apparatus (server) 50 to the portable terminal 60.

### Second Embodiment

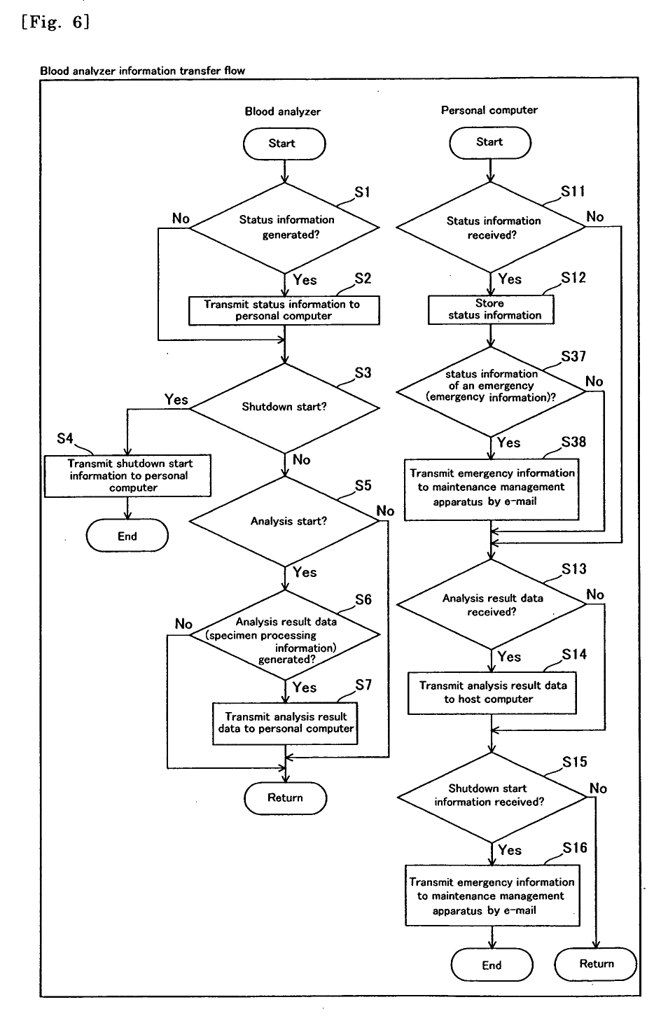

FIG. 6 is a flow chart illustrating the information transmission operation on the blood analyzer side in a second embodiment of the present invention. Described below with reference to FIG. 6 is the case in which not only the emergency information of the specimen smear preparation device 3 but also the emergency information of the blood analyzer 2 is transmitted immediately from the personal computer 4 to the maintenance management apparatus (server) 50 without waiting for shutdown in the second embodiment, which differs from the first embodiment. The apparatus structures of the clinical specimen processing apparatus of the second embodiment is identical to the apparatus structures of the clinical specimen processing apparatus of the first embodiment.

In the second embodiment, the specimen smear preparation device 3 performs the information transmission operations as shown in FIGS. 4 and 5 in the same manner as in the first embodiment. That is, emergency information (emergency error information) of an emergency which is difficult to recover from on the user side is transmitted immediately over a network to the maintenance management apparatus (server) 50 without waiting a predetermined timing, and status information of the specimen smear p[reparation device 3 is transmitted to the maintenance management apparatus (server) 50 when the specimen smear preparation device 3 shuts down.

In the second embodiment, unlike the first embodiment, the blood analyzer 2 performs the information transmission operation shown in FIG. 6. In the flow chart shown in FIG. 6, the processes of steps S1˜S7, steps S11˜S12, and steps S13˜S16 are identical to the information transmission process of the blood analyzer side of the first embodiment shown in FIG. 3. In the second embodiment, in step S37 a determination is made as to whether or not the status information of the blood analyzer 2 stored in the memory 4a of the personal computer 4 is status information for an emergency (emergency information). The status information of an emergency (emergency information) is defined as error information or the like representing a condition which is difficult to deal with on the user side and which cannot be recovered without a visit from the service personnel of the maintenance company.

The type (error code) of status information (emergency information) of emergencies of the blood analyzer 2 are set beforehand in the personal computer 4. When emergency information is determined in step S37, then in step S38 the emergency information of the blood analyzer 2 is transmitted immediately without waiting for shutdown by the controller 4a of the personal computer 4 over the network to the maintenance management apparatus (server) 50 by electronic mail. In this case the emergency information is transmitted as content in the text body of the electronic mail. The mail text body includes, for example, device ID, date, time, and error code.

When it is determined in step S37 that the status information is not status information of an emergency (emergency information), steps S13 and S14 are performed (same steps as in FIG. 3), and in step S15 a determination is made as to whether or not shutdown start information has been received from the blood analyzer 2, and when it determined that shutdown start information has not been received the routine returns to step S11. When it is determined in step S15 that shutdown start information has been received, then in step S16 the controller 4a of the personal computer 4 which received the shutdown start information transmits the status information of the blood analyzer 2 stored in the memory 4b of the personal computer 4 over the network to the maintenance management apparatus (server) 50 by electronic mail. The electronic mail transmission process in this case is identical to the transmission process described in step S16 of FIG. 3, and the controller 4a of the personal computer 4 divides the status information into a plurality of types, creates a plurality of file attachments respectively corresponding to the plurality of types, and attaches the plurality of file attachments to a single electronic mail which is transmitted to the maintenance management apparatus (server) 50.

In the second embodiment, not only emergency information of the specimen smear preparation device 3 but also emergency information of the blood analyzer 2 is transmitted from the personal computer 4 to the maintenance management apparatus (server) 50 without waiting for shutdown, such that not only emergency information of the specimen smear preparation device 3 but also emergency information of the blood analyzer 2 can be transmitted to the maintenance management apparatus (server) 50 in real time. In this way when an abnormality occurs which is difficult to recover from on the user side, whether in the specimen smear preparation device 3 or blood analyzer 2, the maintenance company where the maintenance management device (server) 50 is installed is immediately informed of the abnormality. As a result, maintenance can be performed quickly for not only the specimen smear preparation device 3 but also the blood analyzer 2.

### Third Embodiment

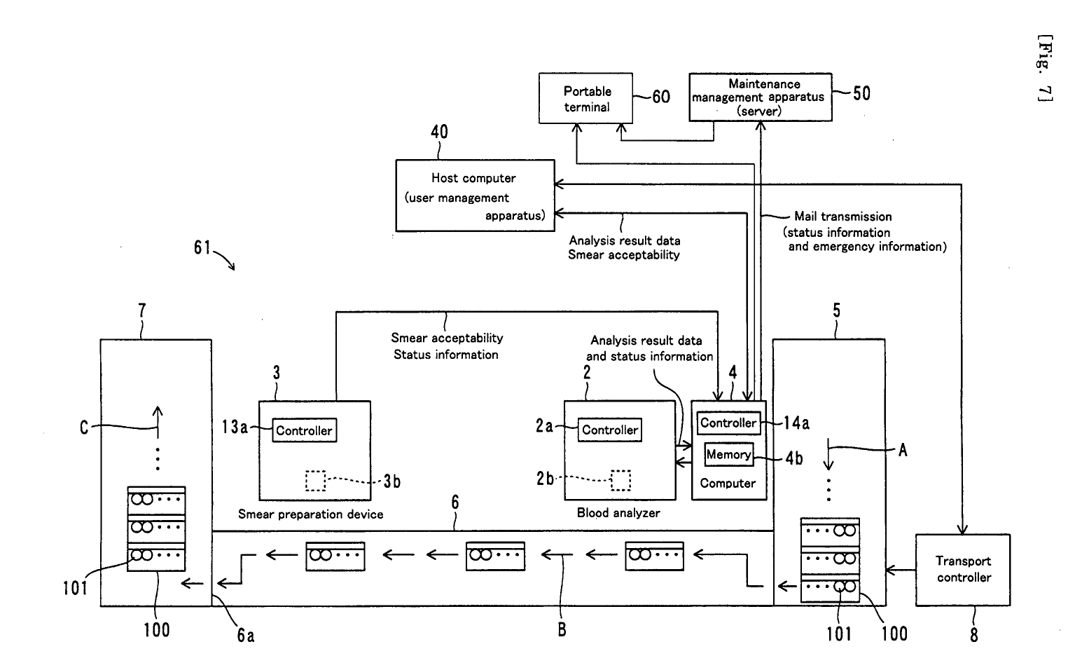

FIG. 7 is a top view showing the general structure of a clinical specimen processing apparatus of a third embodiment of the present invention. Described below with reference to FIG. 7 is the case in which not only the status information of the specimen smear preparation device 3 but also the smear data (specimen processing data) are transmitted from the specimen smear preparation device 3 to the personal computer 4 in the second embodiment, which differs from the first and second embodiments.

In the clinical specimen processing apparatus 61 of the third embodiment shown in FIG. 7, the status information and smear data (smear processing data) of the specimen smear preparation device 3 are transmitted by the controller 3a of the specimen smear preparation device 3 to the personal computer 4. Then, the controller 4a of the personal computer 4 transmits the smear data (smear processing data) from the personal computer 4 to the host computer 40, and transmits the status information of the specimen smear preparation device 3 to the maintenance management apparatus (server) 50.

The transmission of the barcode read by the barcode reader 3b of the specimen smear preparation device 3 and the query as to whether or not to prepare a specimen smear is performed from the specimen smear preparation device 3 to the host computer 40 through the personal computer 4. In other respects the device structure and operation of the third embodiment is identical to the first embodiment.

In the third embodiment, the status information and smear data (specimen processing data) of the specimen smear preparation device 3 are transmitted from the specimen smear preparation device 3 to the personal computer 4, and, therefore, the status information can be easily transmitted to the maintenance management apparatus (server) 50 of the maintenance company, and the specimen processing information of the smear data which includes private information such as patient ID and the like can be easily transmitted to the user host computer 40 using the personal computer 4.

The embodiments disclosed herein are not to be considered as limited to the provided examples. The scope of the present invention is expressed in the scope of the claims and not in the description of the embodiments, and includes all modifications which are within the scope and meaning of equivalence of the claims.

Although the present invention has been described by way of examples applied to clinical specimen processing apparatus including a specimen smear preparation device and blood analyzer in the above embodiments, the present invention is not limited to these applications inasmuch as the invention is also applicable to other clinical specimen processing apparatuss. For example, the present invention is also applicable to clinical specimen processing apparatuss such as immunoassay devices which determine the concentration of cancer-marker antibodies or antigens and infectious disease, blood coagulation measuring devices which measure coagulation function, biochemical analyzers which measure enzyme activity as an indicator of organ function and serum total protein, urine qualitative analyzer for determining the presence/absence of proteins, sugars, and red blood cells in urine specimens, and urine sedimentation analyzers for quantifying red blood cells, white blood cells, epithelial cells, cast, and bacteria.

Although the examples in the above embodiments pertain to transmitting status information from a personal computer to a maintenance management device when shutting down a device, the present invention is not limited to these examples inasmuch as status information also may be transmitted from a personal computer to the maintenance management device when a device is started.

Although the above embodiments have been described in terms of transmitting status information of an emergency (emergency error mail) to a maintenance management apparatus (server) installed at a maintenance company, the present invention is not limited to this arrangement inasmuch as status information of an emergency (emergency error mail) also may be transmitted to a notebook computer and portable telephone of service personnel of the maintenance company.

Although the above embodiments are described in terms of transmitting status information and status information of an emergency (emergency information) to a maintenance management apparatus (server) using electronic mail, the present invention is not limited to this arrangement inasmuch as status information and status information of an emergency (emergency information) also may be transmitted to a maintenance management apparatus (server) by data communication methods other than electronic mail.

1. A clinical specimen processing apparatus connectable over a network to a first management apparatus and a second management apparatus, the clinical specimen processing apparatus comprising:
a specimen processing device for performing predetermined processing of clinical specimens; and
a data processing device comprising a first transmission means for transmitting status information representing the historical operating status of the specimen processing device to the first management device with a predetermined timing; a second transmission means for transmitting emergency information within the status information to the first management device without waiting for the predetermined timing; and a third transmission means for transmitting specimen processing information obtained by predetermined processing of a clinical specimen to the second management device.

2. The clinical specimen processing apparatus of claim 1, wherein the first management apparatus is a maintenance management apparatus installed externally; and the second management apparatus is a user management apparatus installed on the user side.

3. The clinical specimen processing apparatus of claim 1, wherein the specimen processing apparatus is any one among a blood analyzer, specimen smear preparation device, immunoassay device, blood coagulation measuring device, biochemical analyzer, urine qualitative analyzer, and urine sedimentation analyzer.

4. The clinical specimen processing apparatus of claim 1, wherein the first transmission means transmits the status information by first electronic mail and second transmission means transmits the emergency information by second electronic mail.

5. The clinical specimen processing apparatus of claim 4, wherein the first electronic mail has status information as an attached file, and the second electronic mail has emergency information in the mail text body.

6. The clinical specimen processing apparatus of claim 1, wherein the status information includes at least one among an error log representing the abnormality information history of the clinical specimen processing apparatus, operation count representing the number of operations performed by the clinical specimen processing apparatus, and maintenance parts and replacement parts log representing the maintenance parts and replaced parts.

7. A clinical specimen processing apparatus connectable over a network to a management apparatus, the clinical specimen processing apparatus comprising:
a specimen processing device for performing predetermined processing of clinical specimens; and
a data processing device comprising a first transmission means for transmitting a first electronic mail of first information relating to the specimen processing apparatus in a predetermined format to the management apparatus with a predetermined timing; and a second transmission means for transmitting a second electronic mail of predetermined second information within the first information in a format which is different from the first electronic mail to the management apparatus without waiting for the predetermined timing.

8. The clinical specimen processing apparatus of claim 7, wherein the first information is status information representing the historical operating status of the specimen processing apparatus, and the second information is emergency information within the status information.

9. The clinical specimen processing apparatus of claim 8, wherein the first electronic mail has the status information as an attached file, and the electronic second mail has the emergency information in the mail text body.

10. The clinical specimen processing apparatus of claim 8, wherein the status information includes at least one among an error log representing the abnormality information history of the clinical specimen processing apparatus, operation count representing the number of operations performed by the clinical specimen processing apparatus, and maintenance parts and replacement parts log representing the maintenance parts and replaced parts.

11. The clinical specimen processing apparatus of claim 7, wherein the data processing device comprises a third transmission means for transmitting specimen processing information obtained by predetermined processing of a clinical specimen to the second management apparatus.

12. The clinical specimen processing apparatus of claim 7, wherein the second transmission means transmits the second electronic mail to a portable terminal.

13. The clinical specimen processing apparatus of claim 7, wherein the management apparatus transmits the second electronic mail received from the second transmission means to a portable terminal.

14. The clinical specimen processing apparatus of claim 7, wherein the specimen processing apparatus is any one among a blood analyzer, specimen smear preparation device, immunoassay device, blood coagulation measuring device, biochemical analyzer, urine qualitative analyzer, and urine sedimentation analyzer.

15. A clinical specimen processing system connectable over a network to a management apparatus, the clinical specimen processing system comprising:
a first specimen processing device for performing predetermined processing of clinical specimens;
a second specimen processing device for performing predetermined processing of clinical specimen; and
a data processing device comprising a first transmission means for transmitting status information representing the historical operating status of the first specimen processing device to the management device with a predetermined timing; a second transmission means for transmitting emergency information within the status information of the first specimen processing device to the management device without waiting for the predetermined timing; and a third transmission means for transmitting status information representing the historical operating status of the second specimen processing device to the management device with a predetermined timing.

16. The clinical specimen processing system of claim 15 further comprising a second management device, wherein the data processing device further comprises a fourth transmission means for transmitting first specimen processing information obtained by the predetermined processing of a clinical specimen performed by the first specimen processing device to the second management device; and a fifth transmission means for transmitting second specimen processing information obtained by the predetermined processing of a clinical specimen performed by the second specimen processing device to the second management device.

17. The clinical specimen processing system of claim 16, wherein the management apparatus is a maintenance management apparatus installed externally; and the second management apparatus is a user management apparatus installed on the user side.

18. The clinical specimen processing system of claim 15, wherein the first specimen processing device and second specimen processing device are any one among a blood analyzer, specimen smear preparation device, immunoassay device, blood coagulation measuring device, biochemical analyzer, urine qualitative analyzer, and urine sedimentation analyzer.

19. The clinical specimen processing system of claim 15, wherein the first transmission means transmits the status information by first electronic mail and second transmission means, second transmission means transmits the emergency information by second electronic mail and third transmission means transmits the status information by third electronic mail.

20. The clinical specimen processing system of claim 15, wherein the first electronic mail has the status information of the first specimen processing device as an attached file; the second electronic mail has emergency information of the first specimen processing device in the mail text body; and the third electronic mail has status information of the second specimen processing device as an attached file.
